(19) United States
(12) Patent Application Publication
(10) Pub. No.: US 2008/0194641 A1
(43) Pub. Date: Aug. 14, 2008

(54) Pesticidal Mixtures

(75) Inventors: Douglas D. Anspaugh, Apex, NC (US); Nigel Armes, Raleigh, NC (US); David G. Kuhn, Apex, NC (US); Hassan Oloumi-Sadeghi, Raleigh, NC (US)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/915,693

(22) PCT Filed: May 30, 2006
(86) PCT No.: PCT/EP2006/062714

§ 371 Date: Nov. 27, 2007

Related U.S. Application Data
(60) Provisional application No. 60/687,111, filed on Jun. 3, 2005.

(51) Int. Cl. A01N 43/40; A01N 35/10; A01N 47/34; A01P 7/04; A61P 33/00; A01N 53/02

(52) U.S. Cl. 514/341; 514/632; 514/531; 514/590

(57) ABSTRACT

Pesticidal mixtures comprising, as active components 1) a compound of the formula I wherein W is CI or CF₃; X and Y are each independently CI or Br; R¹ is alkyl, alkenyl, alkynyl, or cycloalkyl optionally substituted with 1 to 3 halogens, or alkyl which is substituted by alkoxy; R² and R³ are alkyl or may be taken together to form cycloalkyl optionally substituted by 1 to 3 halogens; R⁴ is H or C₁-C₈-alkyl, or the enantiomers or salts thereof, and a Compound II selected from organo(thio)phosphates, carbamates, pyrethroids, growth regulators, nicotinic receptor agonists/antagonists Compounds, GABA antagonist compounds, macrocyclic lactone insecticides, METI I acaricides, METI II and III compounds, uncoupler compounds, oxidative phosphorylation inhibitor compounds, moulting disrupter compounds, mixed function oxidase inhibitor compounds, sodium channel blocker compounds, and other various pesticide compounds. In synergistically effective amounts, methods for the control of insects or acarids by contacting them or their food supply, habitat, breeding grounds or their locus with mixtures of the compound I with a compound II, a method of protecting plants from attack or infestation by insects or acarids employing mixtures of the compound I with a compound II, and a process for the preparation of a composition for treating, controlling, preventing or protecting a warm-blooded animal or a fish against infestation or infection by insects or acarids which comprises a pesticidally effective amount of a mixture of the compound I with a compound II.

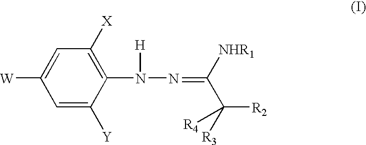

The present invention relates to pesticidal mixtures comprising, as active components

1) a compound of the formula I

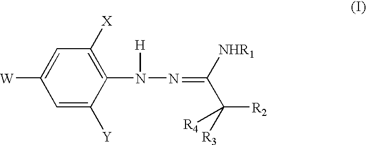

wherein
W is chlorine or trifluoromethyl;
X and Y are each independently chlorine or bromine;
R¹ is C₁-C₆-alkyl, C₃-C₆-alkenyl, C₃-C₆-alkynyl, or
   C₃-C₆-cycloalkyl which is unsubstituted or substituted with 1 to 3 halogen atoms,
   or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl,
or the enantiomers or salts thereof, and
   2) a compound II selected from group A consisting of
A.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
A.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
A.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, taufluvalinate, tefluthrin, tetramethrin, tralomethrin, transfluthrin, profluthrin, dimefluthrin;
A.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentezine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
A.5. Nicotinic receptor agonists/antagonists compounds: clothianidin, dinotefuran, imidacloprid, thiamethoxam, nitenpyram, acetamiprid, thiacloprid; the thiazol compound of formula Γ¹

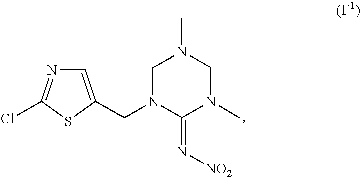

A.6. GABA antagonist compounds: acetoprole, endosulfan, ethiprole, fipronil, vaniliprole, pyrafluprole, pyriprole, the phenylpyrazole compound of formula Γ²

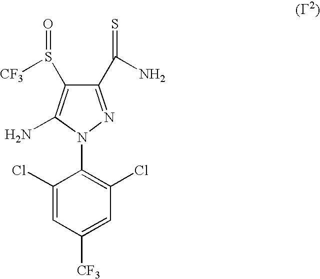

A.7. Macrocyclic lactone insecticides: abamectin, emamectin, milbemectin, lepimectin, spinosad,
A.8. METI I compounds: fenazaquin, pyridaben, tebufenpyrad, tolfenpyrad, flufenerim;
A.9. METI II and III compounds: acequinocyl, fluacyprim, hydramethylnon;
A.10. Uncoupler compounds: chlorfenapyr;
A.11. Oxidative phosphorylation inhibitor compounds: cyhexatin, diafenthiuron, fenbutatin oxide, propargite;
A.12. Moulting disrupter compounds: cyromazine;
A.13. Mixed Function Oxidase inhibitor compounds: piperonyl butoxide;
A.14. Sodium channel blocker compounds: indoxacarb, metaflumizone,
A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet, the aminoisothiazole compounds of formula Γ³,

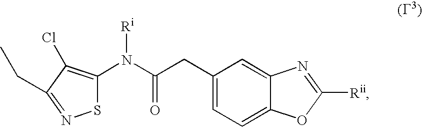

wherein Rⁱ is —CH₂OCH₂CH₃ or H and Rⁱⁱ is CF₂CF₂CF₃ or CH₂CH(CH₃)₃, the anthranilamide compounds of formula Γ⁴

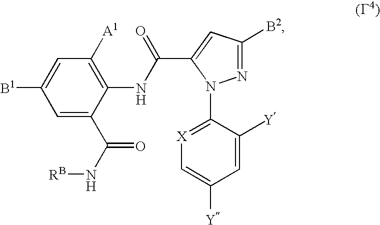

wherein A¹ is CH₃, Cl, Br, I, X is C—H, C—Cl, C—F or N, Y′ is F, Cl, or Br, Y″ is hydrogen, F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂, and the malononitrile compounds 2-(2,2,3,3,4,4,5,5-octafluoropentyl)-2-(3,3,3-trifluoropropyl)malononitrile, 2-(2,2,3,3,4,4,5,5,6,6,7,7-Dodecafluoro-heptyl)-2-(3,3,3-trifluoro-propyl)malononitrile, 2-(3,4,4,4-Tetrafluoro-3-trifluoromethyl-butyl)-2-(3,3,3-trifluoro-propyl)-malononitrile, 2-(3,3,4,4,5,5,6,6,6-Nonafluoro-hexyl)-2-(3,3,3-trifluoro-propyl)-malononitrile, 2,2-Bis-(2,2,3,3,4,4,5,5-octafluoro-pentyl)-malononitrile, 2-(2,2,3,3,4,4,5,5,5-Nonafluoro-pentyl)-2-(3,3,3-trifluoro-propyl)-malononitrile, 2-(2,2,3,3,4,4,4-Heptafluoro-butyl)-2-(2,2,3,3,4,4,5,5-octafluoro-pentyl)-malononitrile and 2-(2,2,3,3,4,4,5,5-Octafluoro-pentyl)-2-(2,2,3,3,3-pentafluoro-propyl)-malononitrile
in synergistically effective amounts.

The present invention also provides methods for the control of insects or acarids by contacting the insect or acarid or their food supply, habitat, breeding grounds or their locus with a pesticidally effective amount of mixtures of the compound I with a compound II.

Moreover, the present invention also relates to a method of protecting plants from attack or infestation by insects or acarids comprising contacting the plant, or the soil or water in which the plant is growing, with a pesticidally effective amount of mixtures of the compound I with a compound II.

This invention also provides a method for treating, controlling, preventing or protecting an animal against infestation or infection by parasites which comprises orally, topically or parenterally administering or applying to the animals a parasiticidally effective amount of a mixture of the compound I with a compound II.

The invention also provides a process for the preparation of a composition for treating, controlling, preventing or protecting a warm-blooded animal or a fish against infestation or infection by insects or acarids which comprises a pesticidally effective amount of a mixture of the compound I with a compound II.

One typical problem arising in the field of pest control lies in the need to reduce the dosage rates of the active ingredient in order to reduce or avoid unfavorable environmental or toxicological effects whilst still allowing effective pest control.

Another problem encountered concerns the need to have available pest control agents which are effective against a broad spectrum of pests.

There also exists the need for pest control agents that combine knock-down activity with prolonged control, that is, fast action with long lasting action.

Another difficulty in relation to the use of pesticides is that the repeated and exclusive application of an individual pesticidal compound leads in many cases to a rapid selection of pests which have developed natural or adapted resistance against the active compound in question. Therefore there is a need for pest control agents that help prevent or overcome resistance.

It was therefore an object of the present invention to provide pesticidal mixtures which solve the problems of reducing the dosage rate and/or enhancing the spectrum of activity and/or combining knock-down activity with prolonged control and/or to resistance management.

We have found that this object is in part or in whole achieved by the combination of active compounds defined at the outset. Moreover, we have found that simultaneous, that is joint or separate, application of a compound I and a compound II or successive application of a compound I and a compound II allows enhanced control of pests compared to the control rates that are possible with the individual compounds.

Compounds of the formula I, their preparation and their action against insect and acarid pests are known (EP-B1 604 798).

The commercially available compounds of the group A may be found in The Pesticide Manual, 13^{th} Edition, British Crop Protection Council (2003) among other publications. Thiamides of formula Γ² and their preparation have been described in WO 98/28279. Aminoisothiazole compounds of formula Γ³ and their preparation have been described in WO 00/06566. Lepimectin is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180.

Anthranilamides of formula Γ⁴ and their preparation have been described in WO 01/70671; WO 02/48137; WO 03/24222, WO 03/15518, WO 04/67528; WO 04/33468; and WO 05/118552. The malononitrile compounds CF₂HCF₂CF₂CF₂CH₂C(CN)₂CH₂CH₂CF₃ (2-(2,2,3,3,4,4,5,5-octafluoropentyl)-2-(3,3,3-trifluoropropyl)malononitrile), CF₃(CH₂)₂C(CN)₂CH₂(CF₂)₅CF₂H (2-(2,2,3,3,4,4,5,5,6,6,7,7-Dodecafluoro-heptyl)-2-(3,3,3-trifluoro-propyl)-malononitrile), CF₃(CH₂)₂C(CN)₂(CH₂)₂C(CF₃)₂F (2-(3,4,4,4-Tetrafluoro-3-trifluoromethyl-butyl)-2-(3,3,3-trifluoro-propyl)-malononitrile), CF₃(CH₂)₂C(CN)₂(CH₂)₂(CF₂)₃CF₃ (2-(3,3,4,4,5,5,6,6,6-Nonafluoro-hexyl)-2-(3,3,3-trifluoro-propyl)-malononitrile), CF₂H(CF₂)₃CH₂C(CN)₂CH₂(CF₂)₃CF₂H (2,2-Bis-(2,2,3,3,4,4,5,5-octafluoro-pentyl)-malononitrile), CF₃(CH₂)₂C(CN)₂CH₂(CF₂)₃CF₃ (2-(2,2,3,3,4,4,5,5,5-Nonafluoro-pentyl)-2-(3,3,3-trifluoro-propyl)-malononitrile), CF₃(CF₂)₂CH₂C(CN)₂CH₂(CF₂)₃CF₂H (2-(2,2,3,3,4,4,4-Heptafluoro-butyl)-2-(2,2,3,3,4,4,5,5-octafluoro-pentyl)-malononitrile) and CF₃CF₂CH₂C(CN)₂CH₂(CF₂)₃CF₂H (2-(2,2,3,3,4,4,5,5-Octafluoro-pentyl)-2-(2,2,3,3,3-pentafluoro-propyl)-malononitrile) have been described in WO 05/63694.

Mixtures, active against pests, of compounds of formula I are described in a general manner in EP-B1 604 798. The use of pesticidal mixtures, active against certain pests, of compounds of formula I with some of the compounds of formula II are described in PCT/EP/04/013687, filed on Dec. 2, 2004 (published as WO 2005/053403). The favourable synergistic effect of these mixtures is not mentioned in these documents but is described herein for the first time.

With regard to their use in the pesticidal mixtures of the present invention, the following compounds of formula I are especially preferred:

Compounds of formula I wherein

W is trifluoromethyl;
X and Y are each independently chlorine or bromine;
R¹ is C₁-C₆-alkyl;
R² and R³ are C₁-C₆-alkyl or may be taken together to form C₃-C₆-cycloalkyl which is substituted by 1 to 2 halogen atoms;
R⁴ is C₁-C₆-alkyl;
or the enantiomers or salts thereof.

Particular preference is given to N-ethyl-2,2-dimethylpropionamide-2-(2,6-dichloro-α,α,α-trifluoro-p-tolyl)hydrazone and N-Ethyl-2,2-dichloro-1-methylcyclopropane-carboxamide, 2-(2,6-dichloro-α,α,α-trifluoro-p-tolyl)hydrazone.

Furthermore, particular preference with respect to the use in the inventive mixtures is given to the compound of formula I-1 (N-ethyl-2,2-dimethylpropionamide-2-(2,6-dichloro-α,α,α-trifluoro-p-tolyl)-hydrazone):

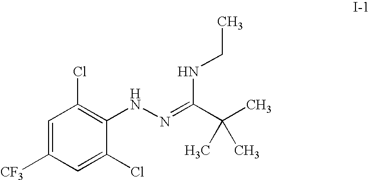

Moreover, particular preference with respect to the use in the inventive mixtures is given to the compound of formula I-2 (N-Ethyl-2,2-dichloro-1-methylcyclopropane-carboxamide-2-(2,6-dichloro-α,α,α-tri-fluoro-p-tolyl)hydrazone):

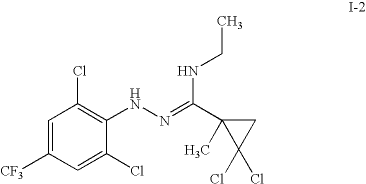

With regard to their use in the pesticidal mixtures of the present invention, the following compounds II of group A are especially preferred:

Preference is given to the compounds II of group A.1.

Especially preferred are the following compounds II of group A.1.: chlorpyrifos, dimethoate, profenofos, terbufos.

Preference is given to the compounds II of group A.2.

Especially preferred are the following compounds II of group A.2.: methomyl, triazamate.

Preference is given to the compounds II of group A.3.

Especially preferred are the following compounds II of group A.3.: bifenthrin, cyhalothrin, cypermethrin, alpha-cypermethrin, deltamethrin, lambda-cyhalothrin, permethrin.

Preference is given to the compounds II of group A.4.

Especially preferred are the following compounds II of group A.4.: flufenoxuron, hexaflumuron, teflubenzuron, novaluron.

Preference is given to the compounds II of group A.5.

Especially preferred are the following compounds II of group A.5.: clothianidin, acetamiprid, imidacloprid, thiamethoxam.

The most preferred compound II of group A.5 is imidacloprid.

Preference is given to the compounds II of group A.6.

Especially preferred are the following compounds II of group A.6.: fipronil, ethiprole.

The most preferred compound II of group A.6 is fipronil.

Preference is given to the compounds II of group A.7.

Especially preferred are the following compounds II of group A.7.: abamectin, emamectin, spinosad.

The most preferred compound II of group A.7. is spinosad.

Preference is given to the compounds II of group A.8.

The most preferred METI I compound of group A.8. is pyridaben.

Preference is given to the compounds II of group A.9.

The most preferred METI II compound of group A.9. is hydramethylnon.

Preference is given to the compound II of group A.10.

Preference is given to the compounds II of group A.11.

Especially preferred are the following oxidative inhibitors of group A.11.: diafenthiuron, fenbutatin oxide.

Preference is given to the compound II of group A.12.

Preference is given to the compound II of group A.13.

Preference is given to the compounds II of group A.14.

Preference is given to the compounds II of group A.15.

Especially preferred are the following compounds of group A.15.: anthranilamide compounds of formula Γ⁴, malononitrile compounds as described in JP 2002 284608, WO 02/89579, WO 02/90320, WO 02/90321, WO 04/06677, WO 04/20399, JP 2004 99597, WO 05/68423, WO 05/68432, or WO 05/63694, especially the malononitrile compounds CF₂HCF₂CF₂CF₂CH₂C(CN)₂CH₂CH₂CF₃ (2-(2,2,3,3,4,4,5,5-octafluoropentyl)-2-(3,3,3-trifluoropropyl)malononitrile), CF₃(CH₂)₂C(CN)₂CH₂(CF₂)₅CF₂H (2-(2,2,3,3,4,4,5,5,6,6,7,7-Dodecafluoro-heptyl)-2-(3,3,3-trifluoro-propyl)-malononitrile), CF₃(CH₂)₂C(CN)₂(CH₂)₂C(CF₃)₂F (2-(3,4,4,4-Tetrafluoro-3-trifluoromethyl-butyl)-2-(3,3,3-trifluoro-propyl)-malononitrile), CF₃(CH₂)₂C(CN)₂(CH₂)₂(CF₂)₃CF₃ (2-(3,3,4,4,5,5,6,6,6-Nonafluoro-hexyl)-2-(3,3,3-trifluoro-propyl)-malononitrile), CF₂H(CF₂)₃CH₂C(CN)₂CH₂(CF₂)₃CF₂H (2,2-Bis-(2,2,3,3,4,4,5,5-octafluoro-pentyl)-malononitrile), CF₃(CH₂)₂C(CN)₂CH₂(CF₂)₃CF₃ (2-(2,2,3,3,4,4,5,5,5-Nonafluoro-pentyl)-2-(3,3,3-trifluoro-propyl)-malononitrile), CF₃(CF₂)₂CH₂C(CN)₂CH₂(CF₂)₃CF₂H (2-(2,2,3,3,4,4,4-Heptafluoro-butyl)-2-(2,2,3,3,4,4,5,5-octafluoro-pentyl)-malononitrile) and CF₃CF₂CH₂C(CN)₂CH₂(CF₂)₃CF₂H (2-(2,2,3,3,4,4,5,5-Octafluoro-pentyl)-2-(2,2,3,3,3-pentafluoro-propyl)-malononitrile), flonicamid, pymetrozine and pyridalyl.

Very preferred compounds of unknown mode of action group A.15. are anthranilamide compounds of formula Γ⁴.

Also, a very preferred compound of Group A.15 is the malononitrile compound CF₂HCF₂CF₂CF₂CH₂C(CN)₂CH₂CH₂CF₃ (2-(2,2,3,3,4,4,5,5-octafluoropentyl)-2-(3,3,3-trifluoropropyl)malononitrile. This compound and its preparation is disclosed in WO 05/63694.

Moreover, especially preferred with regard to their use according to the present invention are mixtures wherein the compound of formula II is selected from the groups A.3. (pyrethroids), A.4. (growth regulators), A.5. (nicotinic receptor agonists/antagonists compounds), A.6. (GABA antagonist compounds), A.7. (macrocyclic lactone insecticides), A.10. (uncoupler compounds), A.14. (sodium channel blocker compounds), or A.15. (various pesticides).

Most preferred are mixtures wherein the compound of formula II is selected from the group A-1 consisting of bifenthrin, cyhalothrin, cypermethrin, alpha-cypermethrin, deltamethrin, lambda-cyhalothrin, permethrin; flufenoxuron, hexaflumuron, teflubenzuron, novaluron; clothianidin, acetamiprid, imidacloprid, thiamethoxam; fipronil, ethiprole; abamectin, emamectin, spinosad; chlorfenapyr; indoxacarb, metaflumizone; anthranilamide compounds of formula Γ⁴, malononitrile compounds as described in JP 2002 284608, WO 02/89579, WO 02/90320, WO 02/90321, WO 04/06677, WO 04/20399, JP 2004 99597, WO 05/68423, WO 05/68432, or WO 05/63694, especially the malononitrile compounds CF₂HCF₂CF₂CF₂CH₂C(CN)₂CH₂CH₂CF₃ (2-(2,2,3,3,4,4,5,5-octafluoropentyl)-2-(3,3,3-trifluoropropyl)malononitrile), CF₃(CH₂)₂C(CN)₂CH₂(CF₂)₅CF₂H (2-(2,2,3,3,4,4,5,5,6,6,7,7-Dodecafluoro-heptyl)-2-(3,3,3-trifluoro-propyl)-malononitrile), CF₃(CH₂)₂C(CN)₂(CH₂)₂C(CF₃)₂F (2-(3,4,4,4-Tetrafluoro-3-trifluoromethyl-butyl)-2-(3,3,3-trifluoro-propyl)-malononitrile), CF₃(CH₂)₂C(CN)₂(CH₂)₂(CF₂)₃CF₃ (2-(3,3,4,4,5,5,6,6,6-Nonafluoro-hexyl)-2-(3,3,3-trifluoro-propyl)-malononitrile), CF₂H(CF₂)₃CH₂C(CN)₂CH₂(CF₂)₃CF₂H (2,2-Bis-(2,2,3,3,4,4,5,5-octafluoro-pentyl)-malononitrile), CF₃(CH₂)₂C(CN)₂CH₂(CF₂)₃CF₃ (2-(2,2,3,3,4,4,5,5,5-Nonafluoro-pentyl)-2-(3,3,3-trifluoro-propyl)-malononitrile), CF₃(CF₂)₂CH₂C(CN)₂CH₂(CF₂)₃CF₂H (2-(2,2,3,3,4,4,4-Heptafluoro-butyl)-2-(2,2,3,3,4,4,5,5-octafluoro-pentyl)-malononitrile) and CF₃CF₂CH₂C(CN)₂CH₂(CF₂)₃CF₂H (2-(2,2,3,3,4,4,5,5-Octafluoro-pentyl)-2-(2,2,3,3,3-pentafluoro-propyl)-malononitrile), flonicamid, pymetrozine and pyridalyl.

Synergistic mixtures of the compound of formula I-1: N-ethyl-2,2-dimethylpropionamide-2-(2,6-dichloro-α,α,α-trifluoro-p-tolyl)hydrazone with one of the pesticides of group A are especially preferred.

Also especially preferred are synergistic mixtures of the compound of formula I-2: N-Ethyl-2,2-dichloro-1-methylcyclopropane-carboxamide, 2-(2,6-dichloro-α,α,α-trifluoro-p-tolyl)hydrazone with one of the pesticides of group A.

When preparing the mixtures, it is preferred to employ the pure active compounds I and II, to which further active compounds, also against harmful fungi or else herbicidal or growth-regulating active compounds or fertilizers can be added.

The mixtures of compounds I and II, or the compounds I and II used simultaneously, that is jointly or separately, exhibit outstanding action against pests from the following orders:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Hellothis armigera, Hellothis virescens, Hellothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana; Trichoplusia ni and Zeiraphera canadensis,
beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica; Sitona lineatus and Sitophilus granaria,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Della coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii; Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata; Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa
thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossularae, Aphis schneideri, Aphis spiraecola, Aphis sambuci, Acyrthosiphon pisum, Aulacorthum solani, Bernisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,
Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni, Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa,
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica, centipedes (Chilopoda), e.g. Scutigera coleoptrata,
millipedes (Diplopoda), e.g. Narceus spp.,
Earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

Moreover, the inventive mixtures are especially useful for the control of Isoptera, Diptera, Blattaria (Blattodea), Hymenoptera, Siphonaptera, and Acarina.

The mixtures according to the invention or the compounds I and II can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable include:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations: 1. Products for dilution with water

### A) Soluble Concentrates (SL, LS)

10 parts by weight of the active compounds are dissolved in water or in a water-soluble solvent. As an alternative, wetters or other auxiliaries are added. The active compound dissolves upon dilution with water.

### B) Dispersible Concentrates (DC)

20 parts by weight of the active compounds are dissolved in cyclohexanone with addition of a dispersant, for example polyvinylpyrrolidone. Dilution with water gives a dispersion.

### C) Emulsifiable Concentrates (EC)

15 parts by weight of the active compounds are dissolved in xylene with addition of calcium dodecylbenzenesulfonate and castor oil ethoxylate (in each case 5% strength). Dilution with water gives an emulsion

### D) Emulsions (EW, EO, ES)

40 parts by weight of the active compounds are dissolved in xylene with addition of calcium dodecylbenzenesulfonate and castor oil ethoxylate (in each case 5% strength). This mixture is introduced into water by means of an emulsifier (Ultraturax) and made into a homogeneous emulsion. Dilution with water gives an emulsion.

### E) Suspensions (SC, OD, FS)

In an agitated ball mill, 20 parts by weight of the active compounds are comminuted with addition of dispersant, wetters and water or an organic solvent to give a fine active compound suspension. Dilution with water gives a stable suspension of the active compound.

### F) Water-Dispersible Granules and Water-Soluble Granules (WG, SG)

50 parts by weight of the active compounds are ground finely with addition of dispersants and wetters and made into water-dispersible or water-soluble granules by means of technical appliances (for example extrusion, spray tower, fluidized bed). Dilution with water gives a stable dispersion or solution of the active compound.

### G) Water-Dispersible Powders and Water-Soluble Powders (WP, SP, WS)

75 parts by weight of the active compounds are ground in a rotor-stator mill with addition of dispersant, wetters and silica gel. Dilution with water gives a stable dispersion or solution with the active compound.

### 2. Products to be Applied Undiluted

### H) Dustable Powders (DP, DS)

5 parts by weight of the active compounds are ground finely and mixed intimately with 95% of finely divided kaolin. This gives a dustable product.

### I) Granules (GR, FG, GG, MG)

0.5 part by weight of the active compounds is ground finely and associated with 95.5% carriers. Current methods are extrusion, spray-drying or the fluidized bed. This gives granules to be applied undiluted.

### J) ULV Solutions (UL)

10 parts by weight of the active compounds are dissolved in an organic solvent, for example xylene. This gives a product to be applied undiluted.

The inventive mixtures can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The inventive mixtures may also be used successfully in the ultra-low-volume process (ULV), it being possible to apply formulations comprising over 95% by weight of active compound, or even to apply the active compound without additives.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). These agents can be admixed with the mixtures according to the invention in a weight ratio of 1:10 to 10:1. For example, the plant(s) may be sprayed with a composition of this invention either before or after being treated with other active ingredients.

The mixtures and methods according to the invention are particularly useful for the control of pests. The inventive mixtures are suitable for efficiently controlling insects and arachnids. They can be applied to any and all developmental stages, such as egg, larva, pupa, and adult.

The pests may be controlled by contacting the target pest, its food supply, habitat, breeding ground or its locus with a pesticidally effective amount of the inventive mixtures or of compositions comprising the mixtures.

“Locus” means a plant, seed, soil, area, material or environment in which a pest is growing or may grow.

In general, “pesticidally effective amount” means the amount of the inventive mixtures or of compositions comprising the mixtures needed to achieve an observable effect on growth, including the effects of necrosis, death, retardation, prevention, and removal, destruction, or otherwise diminishing the occurrence and activity of the target organism. The pesticidally effective amount can vary for the various mixtures/compositions used in the invention. A pesticidally effective amount of the mixtures/compositions will also vary according to the prevailing conditions such as desired pesticidal effect and duration, weather, target species, locus, mode of application, and the like.

The inventive mixtures or compositions of these mixtures can also be employed for protecting plants from attack or infestation by insects or arachnids comprising contacting a plant, or soil or water in which the plant is growing.

In the context of the present invention, the term plant refers to an entire plant, a part of the plant or the propagation material of the plant, that is, the seed or the seedling.

Some of the inventive mixtures have systemic action and can therefore be used for the protection of the plant shoot against foliar pests as well as for the protection of the seed and roots against soil pests.

The compounds I and the compound II can be applied simultaneously, that is jointly or separately, or in succession, the sequence, in the case of separate application, generally not having any effect on the result of the control measures.

The compounds I and the compound II are usually applied in a weight ratio of from 100:1 to 1:100, preferably from 20:1 to 1:50, in particular from 5:1 to 1:20. Depending on the desired effect, the application rates of the mixtures according to the invention are from 5 g/ha to 2000 g/ha, preferably from 50 to 1500 g/ha, in particular from 50 to 750 g/ha.

The inventive mixtures are also suitable for the protection of the seed and the seedlings' roots and shoots, preferably the seeds, against soil pests.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds or after having pregerminated the latter. Preferred are FS formulations.

In the treatment of seed, the application rates of the mixture are generally from 0.1 to 10 kg per 100 kg of seed. The separate or joint application of the compounds I and II or of the mixtures of the compounds I and II is carried out by spraying or dusting the seeds, the seedlings, the plants or the soils before or after sowing of the plants or before or after emergence of the plants.

The invention also relates to the propagation products of plants, and especially the seed comprising, that is, coated with and/or containing, a mixture as defined above or a composition containing the mixture of two active ingredients or a mixture of two compositions each providing one of the two active ingredients. The seed comprises the inventive mixtures in an amount of from 0.1 g to 10 kg per 100 kg of seed.

The inventive mixtures are effective through both contact (via soil, glass, wall, bed net, carpet, plant parts or animal parts), and ingestion (bait, or plant part) and through trophallaxis and transfer.

Preferred application methods are into water bodies, via soil, cracks and crevices, pastures, manure piles, sewers, into water, on floor, wall, or by perimeter spray application and bait.

According to a preferred embodiment of the invention, the inventive mixtures are employed via soil application. Soil application is especially favorable for use against ants, termites, flies, crickets, or cockroaches.

According to another preferred embodiment of the invention, for use against non crop pests such as ants, termites, wasps, flies, mosquitoes, crickets, locusts, or cockroaches the inventive mixtures are prepared into a bait preparation.

The bait can be a liquid, a solid or a semisolid preparation (e.g. a gel). The bait employed in the composition is a product which is sufficiently attractive to incite insects such as ants, termites, wasps, flies, mosquitoes, crickets etc. or cockroaches to eat it. This attractant may be chosen from feeding stimulants or para and/or sex pheromones. Suitable feeding stimulants are chosen, for example, from animal and/or plant proteins (meat-, fish- or blood meal, insect parts, crickets powder, egg yolk), from fats and oils of animal and/or plant origin, or mono-, oligo- or polyorganosaccharides, especially from sucrose, lactose, fructose, dextrose, glucose, starch, pectin or even molasses or honey, or from salts such as ammonium sulfate, ammonium carbonate or ammonium acetate. Fresh or decaying parts of fruits, crops, plants, animals, insects or specific parts thereof can also serve as a feeding stimulant. Pheromones are known to be more insect specific. Specific pheromones are described in the literature and are known to those skilled in the art.

Methods to control infectious diseases transmitted by insects (e.g. malaria, dengue and yellow fever, lymphatic filariasis, and leishmaniasis) with the inventive mixture and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The impregnation of curtains and bednets is mostly done by dipping the textile material into emulsions or dispersions of the insecticide or spraying them onto the nets.

The inventive mixtures and the compositions comprising them can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The inventive mixtures are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant control composition of the present invention is directly applied to the nest of the ants or to its surrounding or via bait contact. The mixtures or compositions of the invention can also be applied preventively to places at which occurrence of the pests is expected.

In the case of soil treatment or of application to the pests dwelling place or nest, the quantity of active ingredients ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or inventive mixture.

For use in bait compositions, the typical content of the inventive mixture is from 0.0001 weight % to 15 weight %, desirably from 0.001 weight % to 5% weight %. The composition used may also comprise other additives such as a solvent of the active material, a flavoring agent, a preserving agent, a dye or a bitter agent. Its attractiveness may also be enhanced by a special color, shape or texture.

For use in spray compositions, the content of the inventive mixture is from 0.001 to 80 weights %, preferably from 0.01 to 50 weight % and most preferably from 0.01 to 15 weight %.

For use in treating crop plants, the rate of application of the inventive mixture may be in the range of 0.1 g to 4000 g per hectare, desirably from 25 g to 600 g per hectare, more desirably from 50 g to 500 g per hectare.

It was also an object of the present invention to provide mixtures suitable for treating, controlling, preventing and protecting warm-blooded animals, including humans, and fish against infestation and infection by pests. Problems that may be encountered with pest control on or in animals and/or humans are similar to those described at the outset, namely the need for reduced dosage rates, and/or enhanced spectrum of activity and/or combination of knock-down activity with prolonged control and/or resistance management.

This invention also provides a method for treating, controlling, preventing and protecting warm-blooded animals, including humans, and fish against infestation and infection by pests of the orders Siphonaptera, Hymenoptera, Hemiptera, Orthoptera, Acarina, Phthiraptera, and Diptera, which comprises orally, topically or parenterally administering or applying to said animals a pesticidally effective amount of mixtures according to the invention.

The invention also provides a process for the preparation of a composition for treating, controlling, preventing or protecting a warm-blooded animal or a fish against infestation or infection by pests of the Siphonaptera, Hymenoptera, Hemiptera, Orthoptera, Acarina, Phthiraptera, and Diptera orders which comprises a pesticidally effective amount of a mixture according to the invention.

The above method is particularly useful for controlling and preventing infestations and infections in warm-blooded animals such as cattle, sheep, swine, camels, deer, horses, poultry, goats, dogs and cats as well as humans.

Infestations in warm-blooded animals and fish including, but not limited to, lice, biting lice, ticks, nasal bots, keds, biting flies, muscoid flies, flies, myiasitic fly larvae, chiggers, gnats, mosquitoes and fleas may be controlled, prevented or eliminated by the mixtures according to the invention.

The inventive mixtures and compositions comprising them are especially suitable for efficiently combating the following pests:

fleas (Siphonaptera), e.g. Ctenocephalidea felis, C. canis, Xenopsylla cheopis, Pulex irritans, Tunga penetrans, and Nosopsyllus fasciatus,
ants, wasps, sawflies (Hymenoptera), e.g. Athalia rosae, Atta cephalotes, Atta sexdens, Atta texana, Crematogaster spp., Hoplocampa minuta, Hoplocampa testudinea, Monomorium pharaonis, Solenopsis geminata, Solenopsis invicta, S. richteri, S. xyloni, Pogonomyrmex barbatus, Pogonomyrmex californicus, Dasymutilla occidentalis, Bombus spp. Vespula squamosa, Paravespula vulgaris, P. pennsylvanica, P. germanica, Dolichovespula maculata, Vespa crabro, Polistes rubiginosa, Camponotus floridanus, and Linepitheum humile,
crickets, grasshoppers, locusts (Orthoptera), e.g. Acheta domestica, Forficula auricularia, Gryllotalpa gryllotalpa Locusta migratoria, Melanoplus bivittatus, Melanoplus femur-rubrum, Melanoplus mexicanus, Melanoplus sanguinipes, Melanoplus spretus, Nomadacris septemfasciata, Schistocerca americana, Schistocerca peregrina, Stauronotus maroccanus and Tachycines asynamorus;
Acarina, e.g. ticks (Ixodida), e.g. Phipicephalus sanguineus, or mites, such as Mesostigmata, e.g. Ornithonyssus bacoti and Dermanyssus gallinae, Prostigmata, e.g. Pymotes tritici, or Astigmata, e.g. Acarus siro;
lice (Phthiraptera), e.g. Pediculus humanus capitis, Pediculus humanus corporis, Pythirus pubis, Haematopinus eurysternus, Haematopinus suis, Linognathus vituli and Solenopotes capillatus;
flies, mosquitoes (Diptera), e.g. Aedes aegypti, Aedes albopictus, Aedes vexans, Anastrepha ludens, Anopheles maculipennis, Anopheles crucinas, An. albimanus, An. Gambiae, An. freeborni, An. leucosphyrus, An. minimus, An. quadrimaculatus, Calliphora vicina, Ceratitis capitata, Chrysomya bezziana, Chrysomya hominivorax, Chrysomya macellaria, Chrysomya bezziana, Chrysops discalis, C. silacea, C. atlanticus, Cochliomyia hominivorax, Contarinia sorghicola, Cordylobia anthropophaga, Culicoides furens, Culex pipiens, Culex nigripalpus, C. quinquefasciatus, C. tarsalis, Culiseta inornata, C. melanura, Dacus cucurbitae, Dacus oleae, Dasineura brassicae, Dermatobia hominis, Fannia canicularis, Gasterophilus intestinalis, Glossina morsitans, Glossina palpalis, G. fuscipes, G. tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestics, Muscina stabulans, Oestrus ovis, Oscinella frit, Pegomya hysocyami, Phorbia antiqua, Phorbia brassicae, Phorbia coarctata, Phlebotomus argentipes, Psorophora columbiae, P. discolor, Prosimuliim mixtum, Rhagoletis cerasi, Rhagoletis pomonella, Sarcophaga haemorrhoidalis, Sarcophaga sp., Simuliim vittatum, Stomoxys calcitrans, Tabanus bovinus, Tabanus atratus, T. lineola, T. similis, Tipula oleracea, and Tipula paludosa
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, C. hemipterus, Reduvius senilis, Triatoma spp., and Arilus critatus.

For oral administration to warm-blooded animals, the mixtures according to the invention may be formulated as animal feeds, animal feed premixes, animal feed concentrates, pills, solutions, pastes, suspensions, drenches, gels, tablets, boluses and capsules. In addition, the mixtures according to the invention may be administered to the animals in their drinking water. For oral administration, the dosage form chosen should provide the animal with 0.01 mg/kg to 100 mg/kg of animal body weight per day of the mixture.

Alternatively, the mixtures according to the invention may be administered to animals parenterally, for example, by intraruminal, intramuscular, intravenous or subcutaneous injection. The mixtures according to the invention may be dispersed or dissolved in a physiologically acceptable carrier for subcutaneous injection. Alternatively, the mixtures according to the invention may be formulated into an implant for subcutaneous administration. In addition the mixtures according to the invention may be transdermally administered to animals. For parenteral administration, the dosage form chosen should provide the animal with 0.01 mg/kg to 100 mg/kg of animal body weight per day of the mixture.

The mixtures according to the invention may also be applied topically to the animals in the form of dips, dusts, powders, collars, medallions, sprays, spot-on and pour-on formulations. For topical application, dips and sprays usually contain 0.5 ppm to 5,000 ppm and preferably 1 ppm to 3,000 ppm of the inventive compounds. In addition, the mixtures according to the invention may be formulated as ear tags for animals, particularly quadrupeds such as cattle and sheep.

The pesticidal action of the compounds and the mixtures can be demonstrated by the experiments below:

Potato plants for Colorado potato beetle and two-leaf cotton plants for tobacco budworm were utilized for bioassays. Excised plant leaves were dipped into 1:1 acetone/water dilutions of the active compounds. After the leaves had dried, they were individually placed onto water-moistened filter paper on the bottoms of Petri dishes. Each dish was infested with 5-7 larvae and covered with a lid. Each treatment dilution was replicated 4 times. Test dishes were held at approximately 27° C. and 60% humidity. Numbers of live and morbid larvae were assessed in each dish at 5 days after treatment application, and percent mortality was calculated.

To determine if an insecticidal mixture was synergistic, Limpel's formula was used:

E=X+Y−XY/100

E=Expected % mortality of the mixture
X=% mortality of compound X, as measured independently
Y=% mortality of compound Y, as measured independently

Synergism was evident if the % observed mortality for the mixture was greater than the % expected mortality.

The test results compiled in the tables 1 and 2 below show that the mixtures according to the invention show a considerable enhanced activity demonstrating synergism compared to the calculated sum of the single activities.

TABLE 1
Activity of mixtures of compound I-1 with
alpha-cypermethrin or meta-flumizone

| | Dose rate | % Mortality of | | % Mortality of | |
|---|---|---|---|---|---|
| Active | [ppm] | Colorado Potato Beetle | | Tobacco Budworm | |
| I-1 | 300 | 20.8 | | 16.0 | |
| | 100 | 0 | | 0 | |
| alpha- | 0.06 | 4.2 | | 8.9 | |
| cypermethrin | | | | | |
| | 0.03 | 0 | | 0 | |
| | | % mortality | % mortality | % mortality | % mortality |
| | | observed | expected | observed | expected |
| I-1 + alpha- | 300 + 0.06 | 33.3 | 24.1 | 50.0 | 23.5 |
| cypermethrin | 300 + 0.03 | 33.3 | 20.8 | 33.3 | 16.0 |
| | 100 + 0.06 | 29.2 | 4.2 | 17.9 | 8.9 |
| | 100 + 0.03 | 13.0 | 0 | 17.9 | 0 |
| metaflumizone | 0.1 | | | 1.8 | |
| | 0.06 | | | 3.3 | |
| I-1 + meta- | 300 + 0.1 | | | 50.0 | 17.5 |
| flumizone | 300 + 0.06 | | | 28.6 | 18.8 |

TABLE 2
Activity of mixtures of compound I-2 with alpha-cypermethrin,
metaflumizone or imidacloprid

| | Dose rate | % Mortality of | | % Mortality of | |
|---|---|---|---|---|---|
| Active | [ppm] | Colorado Potato Beetle | | Tobacco Budworm | |
| I-2 | 100 | | | 12.5 | |
| | 60 | 39.6 | | 5.4 | |
| | 30 | 6.3 | | | |
| alpha- | 0.06 | 4.2 | | 8.9 | |
| cypermethrin | | | | | |
| | 0.03 | 0 | | 0 | |
| | | % mortality | % mortality | % mortality | % mortality |
| | | observed | expected | observed | expected |
| I-2 + alpha- | 100 + 0.06 | | | 75.0 | 20.3 |
| cypermethrin | 100 + 0.03 | | | 60.7 | 12.5 |
| | 60 + 0.06 | 79.2 | 42.1 | 42.9 | 13.8 |
| | 60 + 0.03 | 70.8 | 39.6 | 17.9 | 5.4 |
| | 30 + 0.06 | 45.8 | 10.2 | | |
| | 30 + 0.03 | 45.8 | 6.3 | | |
| metaflumizone | 0.3 | | | 78.6 | |
| | 0.1 | | | 1.8 | |
| I-2 + meta- | 100 + 0.3 | | | 100 | 81.3 |
| flumizone | 100 + 0.1 | | | 67.9 | 14.1 |
| | 60 + 0.3 | | | 100 | 79.8 |
| | 60 + 0.1 | | | 64.3 | 7.1 |
| imidacloprid | 0.06 | 0 | | | |
| I-1 + imidaclo- | 60 + 0.06 | 83.3 | 39.6 | | |
| prid | 30 + 0.06 | 41.7 | 6.3 | | |

1-14. (canceled)

15: A pesticidal mixture comprising
a compound of the formula I: 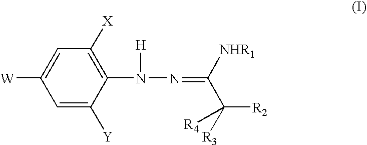
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) Pyrethroids selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 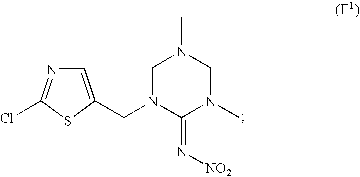
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 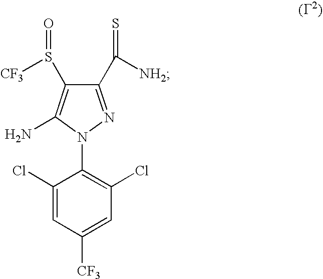
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³ 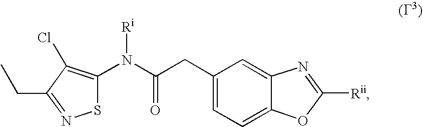
wherein Rⁱ is —CH₂OCH₂CH₃ or H and Rⁱⁱ is CF₂CF₂CF₃ or CH₂CH(CH₃)₃;
an anthranilamide compound of formula Γ⁴: 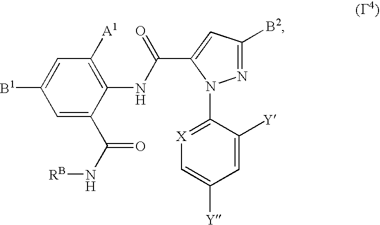
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and
a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
wherein said compound of formula I and said compound II are present in synergistically effective amounts.

16: The pesticidal mixture of claim 15, wherein the compound of formula I is compound I-1:

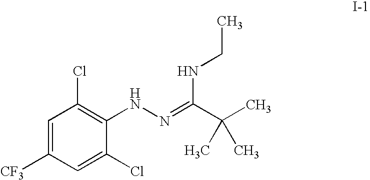

17: The pesticidal mixture of claim 15, wherein the compound of formula I is compound I-2:

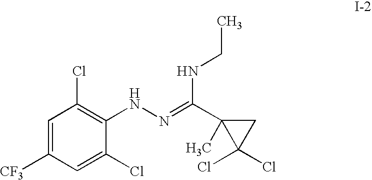

18: The pesticidal mixture of claim 15, wherein compound II is selected from the group A(3), A(4), A(5), A(6), A(7), A(10), A(14), or A(15).

19: The pesticidal mixture of claim 15, wherein compound II is selected from the group consisting of bifenthrin, cyhalothrin, cypermethrin, alpha-cypermethrin, deltamethrin, lambda-cyhalothrin, permethrin, flufenoxuron, hexaflumuron, teflubenzuron, novaluron, clothianidin, acetamiprid, imidacloprid, thiamethoxam, fipronil, ethiprole, abamectin, emamectin, spinosad, chlorfenapyr, indoxacarb, metaflumizone, an anthranilamide compound of formula Γ⁴; a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-Octafluoro-pentyl)-2-(2,2,3,3,3-pentafluoro-propyl)-malononitrile, flonicamid, pymetrozine, and pyridalyl.

20: The pesticidal mixture of claim 15, comprising the compound of formula I and the compound II in a weight ratio of from 100:1 to 1:100.

21: A method for protecting plants from attack or infestation by insects or arachnids comprising:
contacting the plant, or the soil or water in which the plant is growing, with a mixture of:
a compound of the formula I: 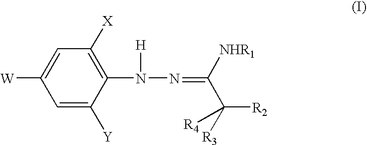
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) a pyrethroid selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 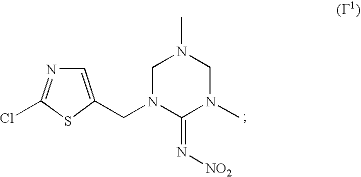
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 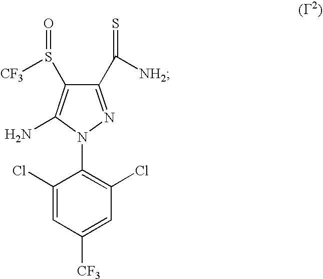
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³: 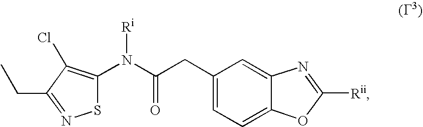
wherein Rⁱ is —CH₂OCH₂CH₃ or H and Rⁱⁱ is CF₂CF₂CF₃ or CH₂CH(CH₃)₃;
an anthranilamide compound of formula Γ⁴: 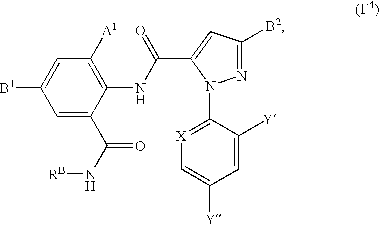
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and
a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
wherein said compound of formula I and said compound II are present in pesticidally and synergistically effective amounts.

22: The method of claim 21, wherein the mixture is applied in an amount of from 5 g/ha to 2000 g/ha.

23: The method of claim 21, wherein the compound of formula I and the compound II are applied simultaneously, that is jointly or separately, or in succession.

24: A method for controlling insects or arachnids comprising contacting an insect or arachnid or their food supply, habitat, breeding grounds or their locus with a mixture of
a compound of the formula I: 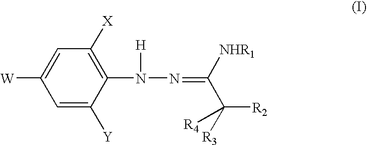
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) a pyrethroid selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 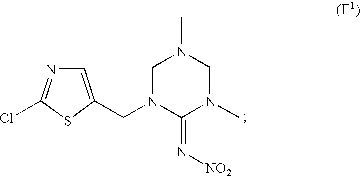
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 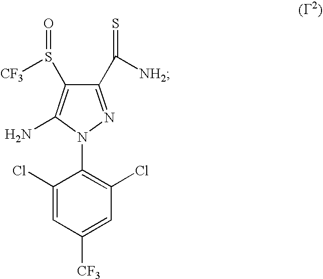
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³: 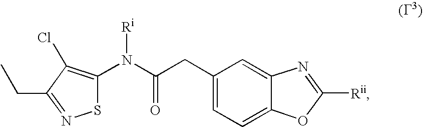
wherein Rⁱ is —CH₂OCH₂CH₃ or H and Rⁱⁱ is CF₂CF₂CF₃ or CH₂CH(CH₃)₃;
an anthranilamide compound of formula Γ⁴: 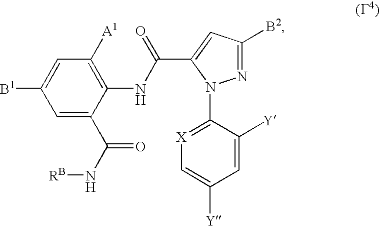
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and
a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
wherein said compound of formula I and said compound II are present in parasiticidally and synergistically effective amounts.

25: The method of claim 24, wherein the mixture is applied in an amount of from 5 g/ha to 2000 g/ha.

26: The method of claim 24, wherein the compound of formula I and the compound II are applied simultaneously, that is jointly or separately, or in succession.

27: A pesticidal composition, comprising a liquid or a solid carrier and a mixture of:
a compound of the formula I: 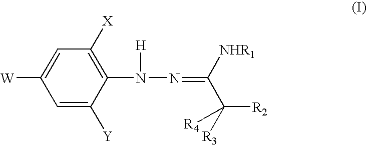
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) a pyrethroid selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 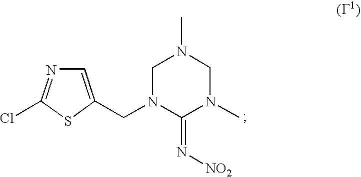
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 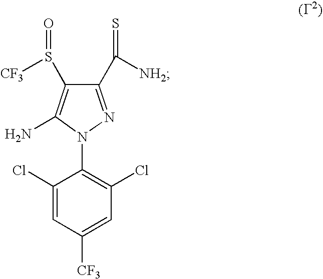
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³: 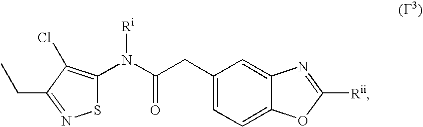
wherein Rⁱ is —CH₂OCH₂CH₃ or H and Rⁱⁱ is CF₂CF₂CF₃ or CH₂CH(CH₃)₃;
an anthranilamide compound of formula Γ⁴: 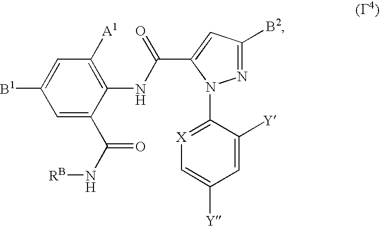
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and
a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
wherein said compound of formula I and said compound II are present in synergistically effective amounts.

28: A method for treating, controlling, preventing or protecting a warm-blooded animal or a fish against infestation or infection by parasites comprising:
orally, topically, or parenterally administering or applying to said animal or fish a mixture of:
a compound of the formula I: 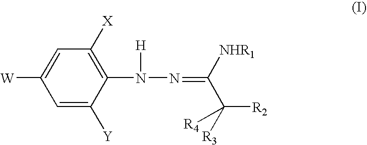
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) a pyrethroid selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 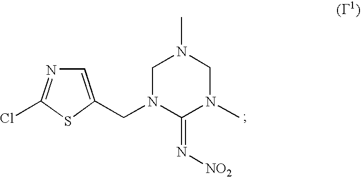
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 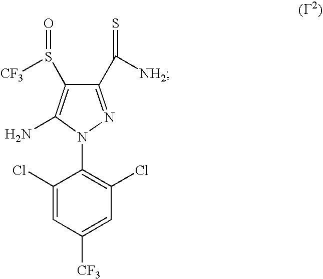
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³: 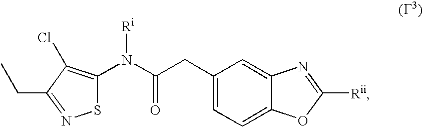
wherein Rⁱ is —CH₂OCH₂CH₃ or H and Rⁱⁱ is CF₂CF₂CF₃ or CH₂CH(CH₃)₃;
an anthranilamide compound of formula Γ⁴: 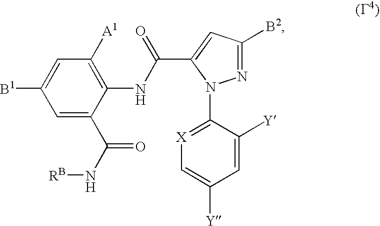
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
wherein said compound of formula I and said compound II are present in parasiticidally and synergistically effective amounts.

29: A process for the preparation of a composition for treating, controlling, preventing or protecting a warm-blooded animal or a fish against infestation or infection by insects or acarids comprising:
combining a pesticidally effective amount of a mixture of:
a compound of the formula I: 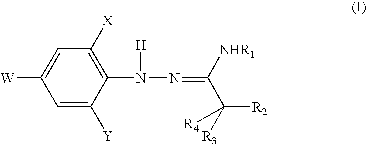
wherein
W is chlorine or trifluoromethyl;
X and Y are each independently chlorine or bromine;
R¹ is C₁-C₆-alkyl, C₃-C₆-alkenyl, C₃-C₆-alkynyl, or C₃-C₆-cycloalkyl which is unsubstituted or substituted with 1 to 3 halogen atoms, or C₂-C₄-alkyl which is substituted by C₁-C₄-alkoxy;
R² and R³ are C₁-C₆-alkyl or may be taken together to form C₃-C₆-cycloalkyl which is unsubstituted or substituted by 1 to 3 halogen atoms;
R⁴ is hydrogen or C₁-C₆-alkyl;
or enantiomers or salts thereof, and
a compound II selected from group A:
A(1) an organo(thio)phosphate selected from the group consisting of: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon;
A(2) a carbamate selected from the group consisting of: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate;
A(3) a pyrethroid selected from the group consisting of: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, and dimefluthrin;
A(4) a growth regulator selected from the group consisting of: a) chitin synthesis inhibitors selected from the group consisting of: benzoylureas, chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, and clofentezine; b) ecdysone antagonists selected from the group consisting of: halofenozide, methoxyfenozide, tebufenozide, and azadirachtin; c) juvenoids selected from the group consisting of: pyriproxyfen, methoprene, and fenoxycarb; and d) lipid biosynthesis inhibitors selected from the group consisting of: spirodiclofen, spiromesifen, and spirotetramat;
A(5) a nicotinic receptor agonist/antagonist selected from the group consisting of: clothianidin, dinotefuran, imidacloprid, thiamethoxam, nitenpyram, acetamiprid, thiacloprid, and a thiazol compound of formula Γ¹: 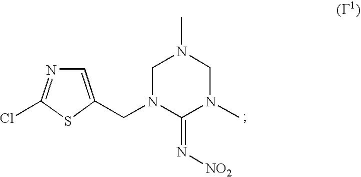
A(6) a GABA antagonist compound selected from the group consisting of: acetoprole, endosulfan, ethiprole, fipronil, vaniliprole, pyrafluprole, pyriprole, and a phenylpyrazole compound of formula Γ²: 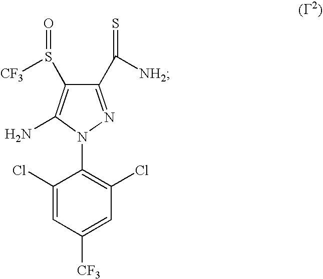
A(7) a macrocyclic lactone insecticide selected from the group consisting of: abamectin, emamectin, milbemectin, lepimectin, and spinosad;
A(8) a METI I compound selected from the group consisting of: fenazaquin, pyridaben, tebufenpyrad, tolfenpyrad, and flufenerim;
A(9) a METI II and III compound selected from the group consisting of: acequinocyl, fluacyprim, and hydramethylnon;
A(10) an uncoupler compound selected from the group consisting of: chlorfenapyr;
A(11) an oxidative phosphorylation inhibitor compound selected from the group consisting of: cyhexatin, diafenthiuron, fenbutatin oxide, and propargite;
A(12) a moulting disrupter compound selected from the group consisting of: cyromazine;
A(13) a Mixed Function Oxidase inhibitor compound selected from the group consisting of: piperonyl butoxide;
A(14) a sodium channel blocker compound selected from the group consisting of: indoxacarb and metaflumizone;
or
A(15) a compound selected from the group consisting of: benclothiaz, bifenazate, cartap, flonicamid, pyridalyl, pymetrozine, sulfur, thiocyclam, flubendiamide, cyenopyrafen, flupyrazofos, cyflumetofen, amidoflumet, an aminoisothiazole compound of formula Γ³: 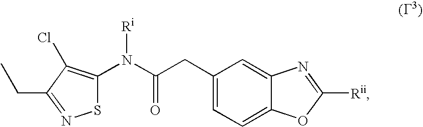
wherein Rⁱ is —CH₂OCH₂CH₃ or H and Rⁱⁱ is CF₂CF₂CF₃ or CH₂CH(CH₃)₃;
an anthranilamide compound of formula Γ⁴: 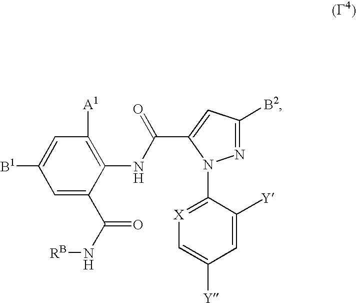
wherein A¹ is CH₃, Cl, Br, or I; X is C—H, C—Cl, C—F or N; Y′ is F, Cl, or Br; Y″ is hydrogen, F, Cl, or CF₃; B¹ is hydrogen, Cl, Br, I, or CN; B² is Cl, Br, CF₃, OCH₂CF₃, or OCF₂H; and R^{B} is hydrogen, CH₃ or CH(CH₃)₂; and
a malononitrile compound selected from the group consisting of 2-(2,2,3,3,4,4,5,5-octafluoropentyl)-2-(3,3,3-trifluoropropyl)malononitrile, 2-(2,2,3,3,4,4,5,5,6,6,7,7-dodecafluoro-heptyl)-2-(3,3,3-trifluoro-propyl)-malononitrile, 2-(3,4,4,4-tetrafluoro-3-trifluoromethyl-butyl)-2-(3,3,3-trifluoro-propyl)-malononitrile, 2-(3,3,4,4,5,5,6,6,6-nonafluoro-hexyl)-2-(3,3,3-trifluoro-propyl)-malononitrile, 2,2-bis-(2,2,3,3,4,4,5,5-octafluoro-pentyl)-malononitrile, 2-(2,2,3,3,4,4,5,5,5-nonafluoro-pentyl)-2-(3,3,3-trifluoro-propyl)-malononitrile, 2-(2,2,3,3,4,4,4-heptafluoro-butyl)-2-(2,2,3,3,4,4,5,5-octafluoro-pentyl)-malononitrile, and 2-(2,2,3,3,4,4,5,5-octafluoro-pentyl)-2-(2,2,3,3,3-pentafluoro-propyl)-malononitrile;
and
a liquid or a solid carrier.
